(19) United States
(12) Patent Application Publication
(10) Pub. No.: US 2025/0332441 A1
(43) Pub. Date: Oct. 30, 2025

(54) Solid-State Lighting With Dual-Frequency Flickering For Visual Stimulation

(71) Applicant: Aleddra Inc., Renton, WA (US)

(72) Inventor: Chungho Hsia, San Jose, CA (US)

(21) Appl. No.: 19/261,931

(22) Filed: Jul. 7, 2025

Related U.S. Application Data
(63) Continuation-in-part of application No. 18/761,773, filed on Jul. 2, 2024.
Application No. 18/761,773 is a continuation-in-part of application No. 18/370,841, filed on Sep. 20, 2023, now Pat. No. 12,382,563.

(51) Int. Cl. A61N 5/06; F21V 3/04; F21V 23/00; F21V 23/02; F21V 23/04; F21Y 105/16; F21Y 115/10; H05B 45/325; H05B 45/46; H05B 45/56; H05B 45/59

(52) U.S. Cl. CPC A61N 5/0622; F21V 3/049; F21V 23/005; F21V 23/02; F21V 23/0407; H05B 45/325; H05B 45/46; H05B 45/56; H05B 45/59; A61N 2005/0629; A61N 2005/0652; F21Y 2105/16; F21Y 2115/10

(57) ABSTRACT

A light-emitting diode (LED) luminaire with dual-frequency visual stimulation flickers, comprising: two sets of LED arrays disposed on a printed circuit board in an interlaced configuration; a pulse-width modulation (PWM) pulse generator; and an LED driving circuit, is configured to replace a conventional luminaire. The PWM pulse generator is configured to generate two modulating signals at respective temporal modulation frequencies. The LED driving circuit is configured to receive the two modulating signals and generate corresponding driving currents to drive the two sets of LED arrays, thereby producing visual stimulation flickers at the two temporal modulation frequencies. The resulting dual-frequency visual stimulation flickers induce a symmetric harmonic frequency perceptible by the human brain, thereby enhancing a benign brain oscillation and endogenously triggering and entraining a gamma-band neural oscillation.

### CROSS-REFERENCE TO RELATED APPLICATIONS

The present disclosure is part of a continuation-in-part (CIP) application of U.S. patent application Ser. No. 18/761,773, filed 2 Jul. 2024, which is a CIP application of U.S. patent application Ser. No. 18/370,841, filed 20 Sep. 2023. Contents of the above-identified application are incorporated herein by reference in its entirety.

### BACKGROUND

### Technical Field

The present disclosure relates to light-emitting diode (LED) luminaires, and more particularly to an LED luminaire configured to emit dual-frequency visual flickers. The dual-frequency flickers are capable of inducing nonlinear visual responses, including the perception of symmetric harmonic frequencies, which may facilitate neural entrainment of brain oscillations, such as gamma-band activity.

### Description of the Related Art

Solid-state lighting from semiconductor light-emitting diodes (LEDs) has received much attention in general lighting applications today. Because of its potential for more energy savings, better environmental protection (with no hazardous materials used), higher efficiency, smaller size, and longer lifetime than conventional incandescent bulbs and fluorescent tubes, the LED-based solid-state lighting will be a mainstream for general lighting in the near future. Meanwhile, advances in LED technologies driven by global initiatives for energy efficiency and clean lighting have led to broader adoption of LED-based systems in various technical fields. Certain research organizations have employed LED lighting in health-related applications, leveraging the intrinsic characteristics of LEDs-such as precise temporal control of light pulses and modulation. These characteristics are particularly relevant in areas including neural stimulation, optogenetics, and visual evoked potential (VEP) studies. In light of this trend, the potential use of temporally modulated light stimuli, including temporal light artifacts, for visual stimulation has gained increasing importance and warrants further technical development and consideration.

According to CIE 17.443 e-ILV, the temporal light artifact (TLA) is an undesired change in visual perception induced by a light stimulus whose luminance or spectral distribution fluctuates with time. A flicker, one of TLA, is a perception of visual unsteadiness for a static observer in a static environment. Furthermore, according to IEEE 1789-2015, flickers are variations in luminance over time (i.e., temporal modulation of light). The health impacts of flicker in LED lighting to consumers have seldom been discussed. Occasionally, when some conventional luminaires or lamps fail resulting in flicker, concurrently introducing seizures in the small percentage of the population that suffers from photosensitive epilepsy. Magnetically ballasted fluorescent lamps or luminaires have flicker issues identified to be related to migraines, headaches, reduced visual performance and comfort, and other possible neurological health issues. When high frequency electronic ballasts become popular, the flicker issues of fluorescent lamps or luminaires diminish. However, a flicker component for such fluorescent lamps or luminaires is between 20% and 25%. For an incandescent lamp and a halogen lamp, the flicker frequency is 120 Hz, and the flicker component is between 15% up to 25%. Compact fluorescent lamps, as energy-saving lamps, have a flicker frequency in a range of 20 kHz to 150 kHz due to a built-in electronic power supply. The flicker component is between 20% and 40%. Since the brightness of LEDs responds instantaneously to an operating current, the flicker frequency and the flicker component depend on a driving current of a power supply used. The flicker component may be between 0% and 100%. The flicker frequency may be from 60 Hz to several hundred kHz, depending on a switching frequency of the power supply used to drive the LEDs. That is, for LED luminaires or lamps, the flicker is primarily determined by the power supply, and some possible health risks are associated with low-frequency modulation of the LEDs.

According to the IEEE Recommended Practice for Evaluating Flicker in Lighting Systems (IEEE Std 1789-2015), potential flicker-induced effects include: (1) neurological symptoms such as photosensitive epileptic seizures; (2) headaches, fatigue, eyestrain, and blurred vision; (3) migraines; (4) reduced visual task performance; (5) increased sensitivity in individuals with autism spectrum disorder, particularly children; (6) perceived motion artifacts (stroboscopic effect); and (7) distraction or annoyance. In view of these considerations, LED driving circuits in power supplies are typically designed to modulate the LED driving current at frequencies considered less likely to cause such effects, and to suppress low-frequency flicker components commonly associated with AC mains power. Notably, certain temporally modulated light stimuli at specific frequencies have also been investigated for their potential to influence neural activity in controlled research settings.

Prior research has reported that exposure to stroboscopic light at 40 Hz for one hour daily over four weeks can entrain gamma-band oscillations in the brain and has shown potential in animal models for investigating Alzheimer's disease. These studies suggest that temporally modulated light stimuli at specific frequencies may influence neural activity associated with cognitive function. However, sustained visual exposure to perceptible 40 Hz stroboscopic flickers may cause visual discomfort or fatigue in humans, particularly with extended daily use. To address this, alternative light delivery mechanisms with imperceptible flicker profiles that can still drive gamma-band neural entrainment are being explored. For example, stimuli at higher frequencies such as 65 Hz, which are less perceptible to human vision, have been investigated for potential use in memory enhancement applications.

Various studies have demonstrated that steady-state visual evoked potentials (SSVEPs), commonly used in Brain-Computer Interface (BCI) systems, can be elicited even when the visual stimulus is imperceptible to the observer at the applied flicker frequency. Rather than employing temporal modulation between light being fully on and off (i.e., a 100% modulation depth), such imperceptible flicker stimuli can be generated by alternating between two light sources-such as two sets of LED arrays. This technique enables temporal modulation at target frequencies without producing perceptible light fluctuations. This approach allows the generation of dual-frequency visual stimulation in a manner that is both visually comfortable and compliant with general lighting standards.

The temporal modulation frequency at which visual flicker becomes imperceptible to a typical human observer is commonly referred to as the critical flicker frequency (CFF), also known as the flicker fusion threshold, which is approximately 48 Hz for brightness-based flicker under standard viewing conditions. Brightness flicker may become perceptible when two modulation signals are in phase and their combined intensity varies over time. The phenomenon in which flicker ceases to be perceptible at or above CFF is known as flicker fusion. In U.S. patent application Ser. No. 18/370,841, filed on Sep. 20, 2023, complementary modulation signals were employed to reduce flicker perception in a system comprising two types of LEDs. However, flicker artifacts may still be perceivable to certain individuals with heightened visual sensitivity, particularly when the modulation frequency is near or below CFF. To address this limitation, modulation frequencies may be increased above the CFF threshold, while incorporating a difference (or beat) frequency component that lies within a benign neural frequency band-such as the theta (4-8 Hz) or alpha (8-13 Hz) ranges. It is believed that such visual stimuli may interact with neural oscillatory dynamics, including the potential to entrain or enhance gamma-band activity, through indirect or endogenous pathways.

The SSVEP approach has been primarily used in communication-focused BCI systems, rather than for therapeutic applications. However, research has suggested that frequency-specific visual stimulation may have potential in the study of neurodegenerative conditions, such as Alzheimer's disease (AD), due to its interaction with neural oscillatory activity. Alzheimer's disease is associated with impairments in visual processing, attention, and neural synchronization. SSVEP-based visual stimulation, particularly using dual-frequency components, has been investigated for its ability to modulate oscillatory brain activity in frequency bands (e.g., theta and alpha) commonly diminished in AD. Some studies have also reported changes in SSVEP amplitude, latency, and frequency coupling as potential indicators of cognitive decline, suggesting a possible role for such stimulation paradigms as non-invasive biomarkers. Dual-frequency stimulation may provide richer and more adaptive modulation patterns than single-frequency approaches, potentially engaging multiple neural circuits simultaneously through frequency interaction. In this context, frequency-specific stimulation-whether visual, electrical, or otherwise—is often referred to as frequency-tuned stimulation, a concept used in both invasive and non-invasive brain stimulation (NIBS) modalities. Frequency tuning involves selecting and optimizing specific stimulation frequencies to modulate neural activity in a targeted and effective manner, a strategy that has been widely explored in neuroscience and clinical research.

### SUMMARY

An LED luminaire with dual-frequency flickers comprising one or more LED arrays, at least one power supply unit, an LED driving circuit, and at least one multi-channel pulse-width modulation (PWM) pulse generator is used to replace a conventional luminaire. The one or more LED arrays may comprise a first set of LED arrays and a second set of LED arrays, each with a forward voltage threshold. Each of the first set of LED arrays and the second set of LED arrays comprises a positive terminal and a negative terminal. The at least one power supply unit is coupled to an alternating current (AC) mains source and configured to convert a line voltage into a primary direct-current (DC) voltage greater than the forward voltage threshold to operate the first set of LED arrays and the second set of LED arrays. The primary DC voltage is configured to apply on the positive terminal of both the first set of LED arrays and the second set of LED arrays with respect to a ground reference. The at least one multi-channel PWM pulse generator is configured to produce a first modulating signal and a second modulating signal respectively at a first predetermined temporal modulation frequency and a second predetermined temporal modulation frequency higher than the first predetermined temporal modulation frequency.

The LED driving circuit comprises at least two modulation circuits configured to produce a first driving current and a second driving current, in response to the first modulating signal and the second modulating signal, to respectively drive the first set of LED arrays and the second set of LED arrays. Each of the at least two modulation circuits comprises an electronic switch configured to be turned on or off in response to the first modulating signal and the second modulating signal. The electronic switch comprises a first terminal and a second terminal. The first terminal is configured to respectively couple to the negative terminal of both the first set of LED arrays and the second set of LED arrays whereas the second terminal is configured to respectively couple to the ground reference.

Each of the first and second modulating signals comprises PWM pulses having a duty cycle of at least 40% and a predetermined period. Each of the first and second driving currents incorporates the respective first and second modulating signals, each associated with its corresponding predetermined period. Therefore, the combined light emitted from the first and second sets of LED arrays exhibits the respective predetermined periods in response to the first and second driving currents. Upon absence of either the first or second LED driving current, the other driving current is configured to increase a duty cycle to at least 75%, thereby maintaining steady combined light output and minimizing perceptible flicker.

The LED luminaire with dual-frequency flickers may further comprise an LED printed circuit board, and a light diffuser disposed in front of the LED printed circuit board. Each of the first set of LED arrays and the second set of LED arrays may comprise a plurality of LED arrays arranged respectively in two interlaced fields, one occupying odd-numbered rows and the other occupying even-numbered rows. The plurality of LED arrays are mounted on the LED printed circuit board and are configured to collectively emit a combined light, which is optically blended by the light diffuser to produce a uniform illumination, thereby visually unifying the dual-frequency flickers emitted from the odd-numbered and even-numbered rows into a single perceived field.

It is important to note that the first predetermined temporal modulation frequency and the second predetermined temporal modulation frequency must be selected such that the resulting beat frequency between them remains imperceptible to the human eye. When appropriately chosen, the beat frequency may occasionally align with either the first or second modulation frequency in phase yet still remains visually undetectable due to its high temporal characteristics. For example, the first predetermined temporal modulation frequency may be nominally 80 Hz, and the second may be nominally 120 Hz, resulting in a beat frequency of 40 Hz, but not perceived visually.

Numerous studies on the visual cortex and gamma rhythms across various brain regions suggest that the primary visual cortex (V1) plays a critical role in visual information processing. Notably, three narrowband gamma rhythms in V1 are associated with the processing of distinct spatial frequency (SF) components and originate from different neural circuits. The low gamma (LG; 25-40 Hz) rhythm is generated in the superficial layers of V1 and is responsive to higher spatial frequencies than those preferred by spike activity. In contrast, both the medium gamma (MG; 40-65 Hz), which is generated cortically, and the high gamma (HG; 65-85 Hz), which originates subcortically, are more sensitive to lower spatial frequency inputs.

Importantly, each of these gamma rhythms is generated by distinct neural mechanisms. Studies indicate that the HG component arises from subcortical regions, while LG and MG are primarily generated through cortical processes. This differentiation helps reconcile conflicting interpretations from earlier research. Further evidence reveals that the MG rhythm is strongest in the input layers of V1, whereas LG peaks in the output layers.

Neural circuits supporting these gamma rhythms involve diverse interneuron types, each with unique biophysical properties and connectivity patterns. In the hippocampus, for example, some neurons preferentially synchronize with the peaks of 4-8 Hz theta oscillations, generating resonance within the theta band. Gamma oscillations (30-90 Hz) are broadly associated with neural coding, inter-regional communication, and cognitive functions such as memory and attention.

When flicker information from natural images enters the visual system via the retina, it may be decomposed into multiple frequency channels within the superficial layers of V1. For example, an 80 Hz visual modulation can drive corresponding 80 Hz oscillatory activity in the subcortical visual system. This input, when combined with internally generated oscillations at approximately 55 Hz in the V1 input layer, may undergo further transformation-potentially being suppressed or reshaped in the output layer into a 40 Hz signal. The 40 Hz frequency falls within the intrinsic oscillatory range (25-50 Hz) observed in the human subiculum.

Gamma oscillations are essential for encoding and retrieving memories. These rhythmic neuronal activities depend on the integrity of interneuron networks, which can be disrupted by factors such as inflammation, oxidative stress, or metabolic imbalance. Gamma activity facilitates synchronization and communication between brain regions, supporting perception, motor control, memory, and emotional regulation. Abnormal gamma rhythms have been linked to central nervous system disorders, including Alzheimer's disease, Parkinson's disease, and schizophrenia.

Recent evidence suggests that gamma entrainment-via sensory stimulation at gamma frequencies—may provide neuroprotective benefits. From both physiological and pathological perspectives, gamma oscillations are increasingly regarded as viable targets for therapeutic intervention in neuropsychiatric disorders. Notably, 40 Hz (a medium gamma frequency) is a subharmonic of 80 Hz (a high gamma frequency), suggesting a resonance mechanism that could underlie the therapeutic potential of dual-frequency visual stimuli. Stimuli operating within these frequency ranges may enhance brain health through internal resonance and improved communication across cortical and subcortical networks.

In an embodiment according to the present disclosure, both the first and second predetermined temporal modulation frequencies are selected to be higher than CFF, rendering the flicker imperceptible to the human eye. However, despite the absence of conscious flicker perception, the underlying modulation remains detectable by the human visual system at the neural level. As such, the imperceptible visual stimuli are processed by the brain, which internally demodulates the high-frequency lighting signals into a benign neural stimulus. This endogenous processing entrains gamma oscillations within the brain without inducing perceptible flicker, thereby minimizing visual fatigue, eyestrain, and associated discomfort while still providing effective neural stimulation.

### BRIEF DESCRIPTION OF THE DRAWINGS

Non-limiting and non-exhaustive embodiments of the present disclosure are described with reference to the following figures, wherein like names refer to like parts but their reference numerals differ throughout the various figures unless otherwise specified. Moreover, in the section of detailed description of the invention, any of a “primary”, a “secondary”, a “first”, a “second”, and so forth does not necessarily represent a part that is mentioned in an ordinal manner, but a particular one.

FIG. 1 is an embodiment of an LED luminaire according to the present disclosure.

FIG. 2 shows an arrangement of LED arrays on an LED printed circuit board according to the present disclosure.

FIG. 3 illustrates an optical arrangement according to the present disclosure.

FIG. 4 shows example waveforms of modulating signals for dual-frequency flicker stimuli according to the present disclosure.

FIG. 5 shows an example of a Fast Fourier Transform (FFT) spectrum of a flicker stimulus according to the present disclosure.

FIG. 6 illustrates various waveforms in the embodiment according to the present disclosure.

FIG. 7 illustrates additional waveforms in the embodiment according to the present disclosure.

### DETAILED DESCRIPTION OF VARIOUS EMBODIMENTS

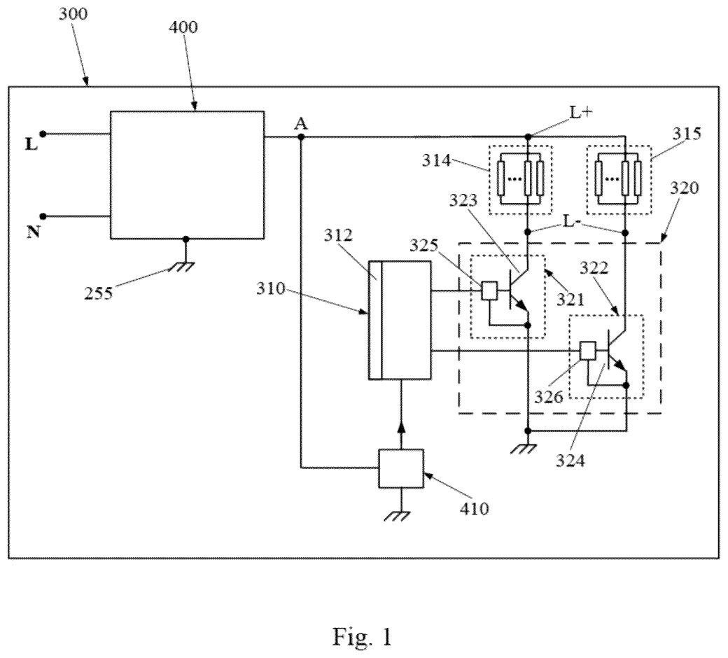

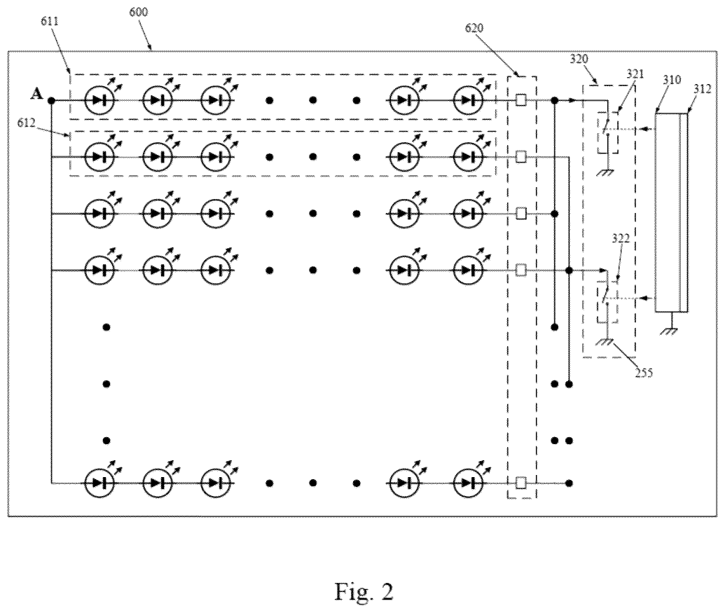

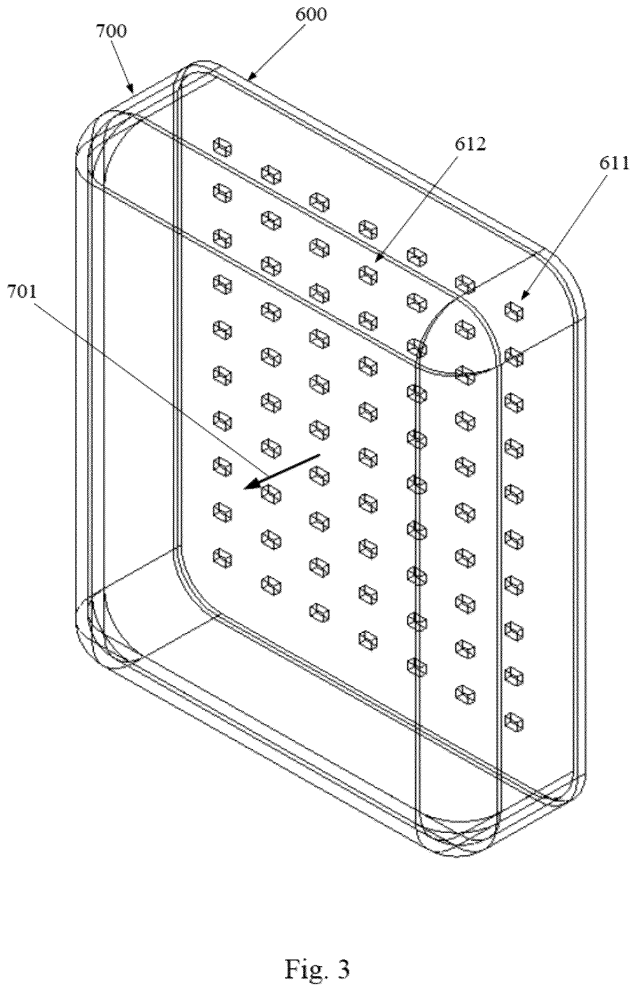

FIG. 1 is an embodiment of an LED luminaire with dual-frequency flickers according to the present disclosure. FIG. 2 shows an arrangement of LED arrays on an LED printed circuit board according to the present disclosure. FIG. 3 illustrates an optical arrangement according to the present disclosure. Referring to FIGS. 1˜3, the LED luminaire 300 with dual-frequency flickers comprises a first set of LED arrays 314 and a second set of LED arrays 315, each with a forward voltage. The LED luminaire 300 with dual-frequency flickers further comprises an LED printed circuit board 600, and a light diffuser 700 disposed in front of the LED printed circuit board 600, facing the first and second set of LED arrays 314 and 315. In FIGS. 2˜3, each of the first and the second set of LED arrays 314 and 315 comprises a plurality of LED arrays arranged respectively in two interlaced fields and mounted on the LED printed circuit board 600, one occupying odd-numbered rows 611 and the other occupying even-numbered rows 612. The plurality of LED arrays mounted on the odd-numbered rows 611 and the even-numbered rows 612 are configured to collectively emit a combined light, which is optically blended by the light diffuser 700 to produce a uniform illumination (FIG. 3), thereby visually unifying the dual-frequency flickers emitted from the odd-numbered and even-numbered rows into a single perceived field. In FIG. 3, the light diffuser 700 is facing the plurality of LED arrays mounted on the odd-numbered rows 611 and the even-numbered rows 612, etc. with its surface normal 701 in an illumination direction. For clarity, a plurality of the odd-numbered rows 611 and the even-numbered rows 612 are only drawn as two single rows. Also, to make the illustration clear, the diffuser 700 is deliberately drawn as transparent though it is opaque. Also note that each of the first set of LED arrays 314 and the second set of LED arrays 315 may further comprise a plurality of current-limiting resistors 620 (as shown in FIG. 2) configured to respectively couple to each of the plurality of LED arrays and to prevent thermal runaway or destruction of the plurality of LED arrays from excess current.

In FIG. 1, the LED luminaire 300 with dual-frequency flickers further comprises at least one power supply unit 400, at least one multi-channel pulse-width modulation (PWM) pulse generator 310, and an LED driving circuit 320. The LED luminaire 300 with dual-frequency flickers further comprises at least two electrical conductors “L” and “N” configured to couple to an alternating current (AC) mains source and configured to convert a line voltage into a primary direct-current (DC) voltage greater than the forward voltage. Each of the first set of LED arrays 314 and the second set of LED arrays 315 further comprises a positive terminal (L+) and a negative terminal (L−). The primary DC voltage is configured to apply on a port denoted as “A”, further on the positive terminal of both the first set of LED arrays 314 and the second set of LED arrays 315 with respect to a ground reference 255.

The at least one multi-channel PWM pulse generator 310 is configured to produce a first modulating signal and a second modulating signal respectively at a first predetermined temporal modulation frequency and a second predetermined temporal modulation frequency higher than the first predetermined temporal modulation frequency. In FIGS. 1˜2, the LED driving circuit 320 comprises at least two modulation circuits 321 and 322 configured to produce a first driving current and a second driving current, in response to the first modulating signal and the second modulating signal, and to respectively drive the first set of LED arrays 314 and the second set of LED arrays 315. The at least two modulation circuits 321 and 322 respectively comprise an electronic switch 323 and 324, each comprising a first terminal and a second terminal. The first terminal is configured to respectively couple to the negative terminal of both the first set of LED arrays 314 and the second set of LED arrays 315 whereas the second terminal is configured to respectively couple to the ground reference 255.

In FIG. 1, the LED luminaire 300 with dual-frequency flickers may further comprise a positive voltage regulator 410 configured to convert the primary DC voltage into a secondary DC voltage that is positive relative to the ground reference 255 and stable enough to operate the at least one multi-channel PWM pulse generator 310, producing the first and second modulating signals to reliably modulate the first and second driving currents. In FIG. 1, each of the electronic switches 323 and 324 is configured to control the plurality of LED arrays in response to the first modulating signal and the second modulating signal. Each of the electronic switches 323 and 324, shown in FIG. 1, is implemented as a bipolar junction transistor (BJT), although a metal-oxide-semiconductor field-effect transistor (MOSFET) may also be used. In FIG. 1, each of the at least two modulation circuits 321 and 322 further comprises a respective resistor network (325, 326) configured to provide appropriate biasing for the corresponding electronic switches 323 and 324, thereby facilitating the generation of the first and second modulating signals with a high signal-to-noise ratio. In FIGS. 1˜2, the at least one multi-channel PWM pulse generator 310 comprises a human interface 312 configured to receive user-defined commands for selecting combinations of modulation frequencies and tuning parameters, thereby enabling personalized modulation of brain rhythms, enhancing neural synchronization and entrainment, and targeting specific cognitive states or neurological biomarkers for therapeutic purposes.

A combined emission from the one or more LED arrays 314 and 315, functioning as two flicker stimuli, is configured to stimulate the visual nerves in the human brain and, consequently, to entrain neuronal oscillations that underlie temporal coordination of neuronal processing. Synchronization of these oscillations facilitates neural communication across distributed brain regions, supporting a variety of sensory, motor, and cognitive functions.

It is well established that neuronal oscillations at different frequencies interact via cross-frequency coupling (CFC), a fundamental property of neural dynamics in which distinct frequency bands exhibit specific coupling relationships. A prominent form of CFC is phase-amplitude coupling (PAC), where the phase of a slower oscillation modulates the amplitude (or power) of a faster one. Examples include alpha (8-12 Hz) phase to gamma (>30 Hz) power coupling in humans, and theta (4-8 Hz) phase to gamma coupling in the rat hippocampus

In the disclosed system, the combined light with the first and second predetermined temporal modulation frequencies may be perceived by the human brain as a difference frequency, particularly if the resulting beat falls within the alpha or theta band. An advantage of this approach is that the light remains perceptually flicker-free if both source modulation frequencies are above the critical flicker fusion threshold-thus eliminating the need for heterochromatic flicker or invisible spectral flicker (ISF) techniques.

Additionally, the application of the second driving current to the second set of LED arrays 315 may be temporally offset from the first driving current applied to the first set of LED arrays 314, thereby introducing a phase difference or time delay between the two modulation signals. This temporal offset may result in the brain perceiving one modulation frequency as being modulated relative to the other, leading to the induction of alpha or theta band oscillations. In such a case, gamma power may be modulated by ongoing theta or alpha phase activity-a robust phenomenon known to coordinate hippocampal and cortical neuronal processing.

This PAC phenomenon has been observed in the human medial temporal lobe and neocortex through intracranial recordings and is also evident in the visual systems of both humans and non-human primates. For example, alpha-gamma coupling has been implicated in mechanisms that prioritize visual processing. Recent primate studies show that alpha activity reflects feedback processing, whereas gamma reflects feedforward processing. Laminar recordings in the primary visual cortex (V1) indicate that alpha oscillations originate in the superficial and deep cortical layers and propagate inward, while gamma oscillations originate in granular layers and propagate outward. Moreover, dual-frequency stimulation-such as 16.4 Hz and 19.1 Hz—has been recently explored to induce CFC, generate beat frequencies (e.g., symmetric harmonics), and engage more complex or resonant neural circuits for enhanced brain stimulation.

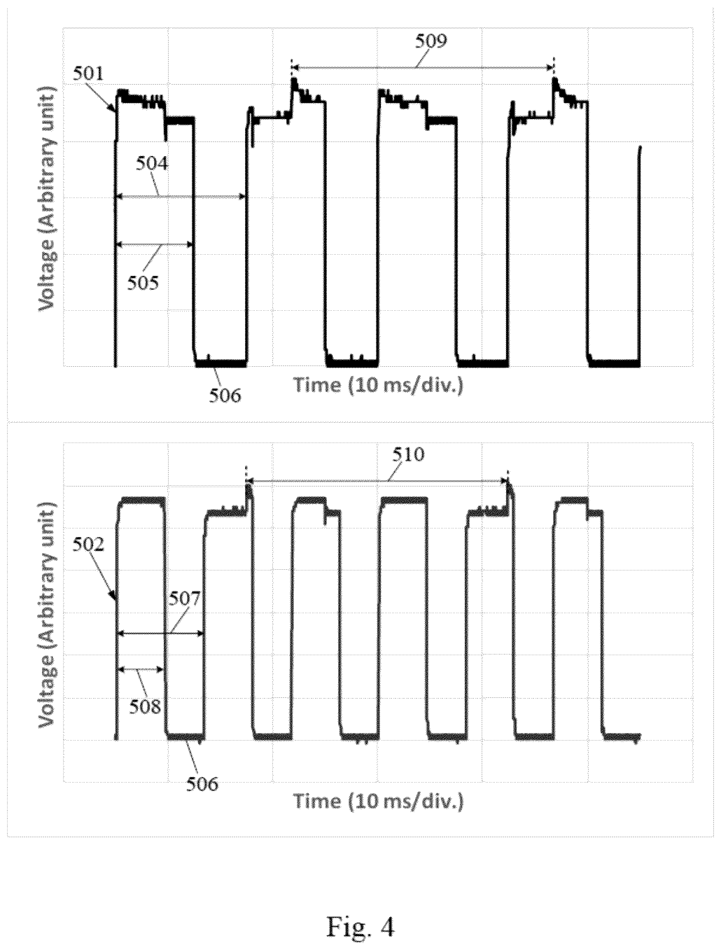

FIG. 4 illustrates example waveforms of modulating signals corresponding to dual-frequency flicker stimuli according to the present disclosure. In this figure, waveforms 501 and 502 represent a first flicker stimulus and a second flicker stimulus, respectively, each generated in response to a corresponding first and second driving current. The waveform 501, referenced to ground voltage 506, has a period 504 corresponding to the first predetermined temporal modulation frequency. Its switch-on duration, indicated by interval 505, reflects an on-time. The duty cycle of waveform 501 is approximately 59%.

Similarly, the waveform 502, also referenced to ground voltage 506, has a period 507 corresponding to the second predetermined temporal modulation frequency, with a switch-on duration 508. The duty cycle of the waveform 502 is approximately 54%. Notably, both the waveforms 501 and 502 exhibit periodic transient anomalies or “glitches”—e.g., within an interval 509 of the waveform 501 and an interval 510 of the waveform 502-which correspond to a beat frequency arising from the interaction between the first and second temporal modulation frequencies. These glitches reflect the phase interference pattern characteristic of dual-frequency modulation and may be associated with the perceived or neural effect of the beat frequency.

As illustrated in FIG. 4, each of the first and second modulating signals comprises PWM pulses having a duty cycle of at least 40% and a predetermined period. Each of the first and second driving currents incorporates the respective first and second modulating signals, each associated with its corresponding predetermined period. The combined light emitted from the first and second sets of LED arrays 314 and 315 exhibits the respective predetermined periods in response to the first and second driving currents. However, upon absence of either the first or second LED driving current, the other driving current is configured to increase a duty cycle to at least 75% (not shown), thereby maintaining steady combined light output and minimizing perceptible flicker.

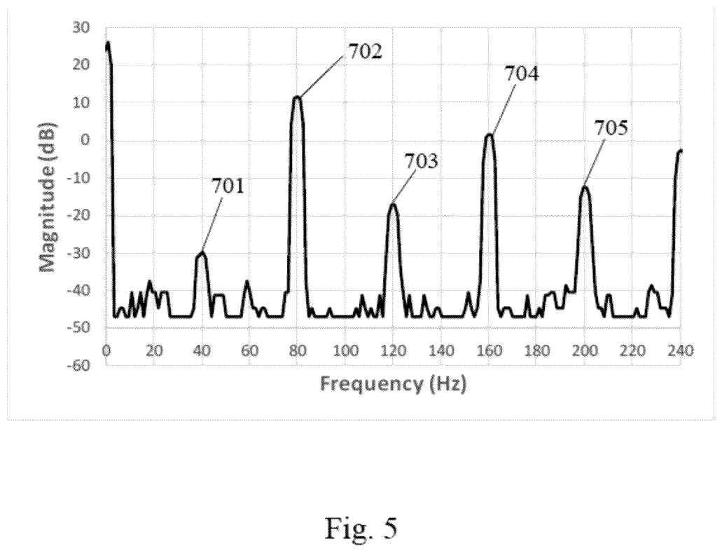

FIG. 5 shows an example Fast Fourier Transform (FFT) of the first flicker stimulus presented in FIG. 4, according to the present disclosure. In FIG. 5, the horizontal axis represents frequency (Hz), and the vertical axis represents magnitude (dB). A series of frequency spikes-701, 702, 703, 704, and 705—are observed, corresponding to 40 Hz, 80 Hz, 120 Hz, 160 Hz, and 200 Hz, respectively.

As previously described, the LED driving circuit 320 is configured to generate two driving currents in response to two corresponding modulating signals at the first and second predetermined temporal modulation frequencies-80 Hz and 120 Hz, respectively—to drive two sets of LED arrays 314 and 315. Both frequencies are above CFF, rendering the stimuli imperceptible to human vision.

Due to potential frequency pre-mixing within the LED driving circuit 320, each of the LED driving currents may contain components of both modulation frequencies (80 Hz and 120 Hz). The modulation circuits include at least two electronic switches, implemented using bipolar junction transistors (BJTs), each biased with a voltage significantly greater than the typical base-emitter voltage (VBE) of 0.7 V. Specifically, the positive voltage regulator 420 supplies an operating voltage (e.g., 5 V) to power the at least one multi-channel PWM pulse generator 310 and to properly bias the BJTs within the modulation circuits 321 and 322. When biased with a signal as high as 5 V-well above the VBE threshold for silicon-based NPN and PNP transistors—the BJTs operate in a nonlinear regime. This nonlinear operation leads to interaction (i.e., mixing) between the first and second modulating signals, resulting in the generation of intermodulation components and harmonic distortion. The resulting distorted waveform can be spectrally decomposed into its fundamental frequencies and associated harmonics.

Specifically, switching activity at 80 Hz and 120 Hz induces electrical and magnetic coupling between the first and second LED driving currents, producing both a beat frequency at 40 Hz (seen as spike 701) and a sum frequency at 200 Hz (spike 705). The beat frequency 40 Hz is the difference between the two carrier frequencies, while the 160 Hz component (spike 704) may arise as a second harmonic of the 80 Hz modulation.

Importantly, although both the 80 Hz and 120 Hz flicker stimuli are imperceptible to the human eye, nonlinear mixing within the LED driving circuit 320 produces a beat frequency at 40 Hz, which is neurologically effective. This 40 Hz beat frequency can entrain gamma-band neuronal oscillations in the brain, potentially reducing eyestrain, visual discomfort, and enhancing cognitive and perceptual functions.

Specifically, the emergence of such a beat frequency or symmetric harmonic (e.g., 40 Hz) is attributed to nonlinear operations within the driving circuitry-such as the transistor-based switching behavior described earlier. In contrast, neural processing in the brain is also inherently nonlinear and is capable of generating intermodulation components and symmetric harmonics independently in response to external stimuli. This dual nonlinearity-electronic and neural—may reinforce the perception and effectiveness of beat frequency-based neural entrainment.

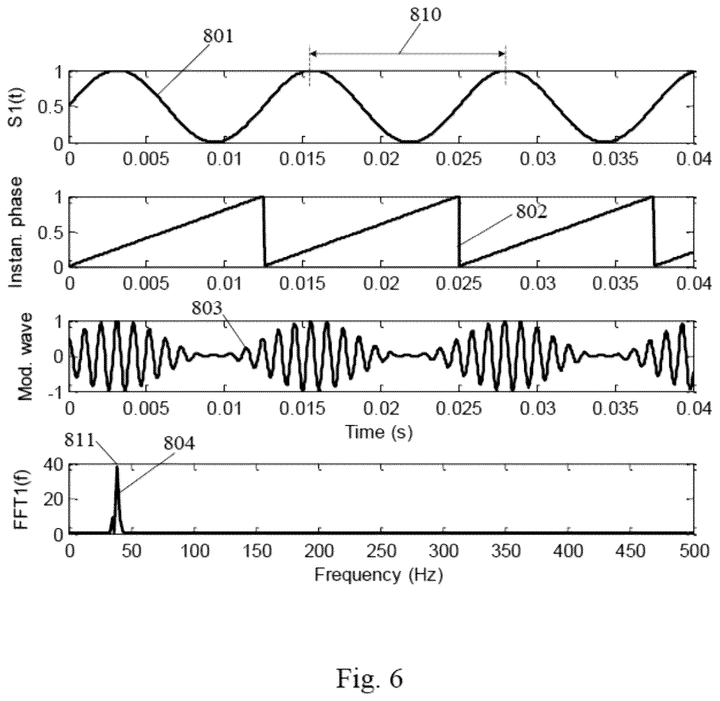

FIG. 6 illustrates example waveforms associated with the embodiment described in the present disclosure. In this figure, it is assumed that both the first and second modulating signals are sinusoidal. The horizontal axes of waveforms 801, 802, and 803 represent time (seconds), whereas the horizontal axis of waveform 804 represents frequency (Hz). In the waveform 801, a modulating signal S1(t) at the first temporal modulation frequency of 80 Hz is shown, with a period 810 corresponding to this frequency. The waveform 802 illustrates the instantaneous phase of S1(t), which is configured to modulate a carrier at the second temporal modulation frequency of 120 Hz. The resulting modulated waveform is shown in the waveform 803. This waveform contains both an upper sideband and a lower sideband, corresponding respectively to the sum (200 Hz) and difference (40 Hz) of the two input frequencies (80 Hz and 120 Hz). When this modulated wave is subjected to demodulation-either electronically or via neural processing-a beat frequency emerges at 40 Hz, as seen at frequency spike 811 in the Fast Fourier Transform FFT1(f) shown in waveform 804.

In contrast, a similar frequency spike at 40 Hz may also arise in SSVEP-based stimulation when using 80 Hz and 120 Hz flicker inputs. In that case, however, the 40 Hz gamma-band response is endogenous, generated by nonlinear neural processing in the brain in response to dual-frequency stimulation. Simultaneously, a higher-frequency stimulation (e.g., 80 Hz) can entrain endogenous gamma oscillations. Thus, the brain responds not only to the beat frequency (e.g., 40 Hz) but also to subharmonic components (e.g., a 40 Hz response elicited by an 80 Hz flicker). This dual entrainment-through both beat frequencies and harmonic coupling-enables flexible, targeted neuromodulation.

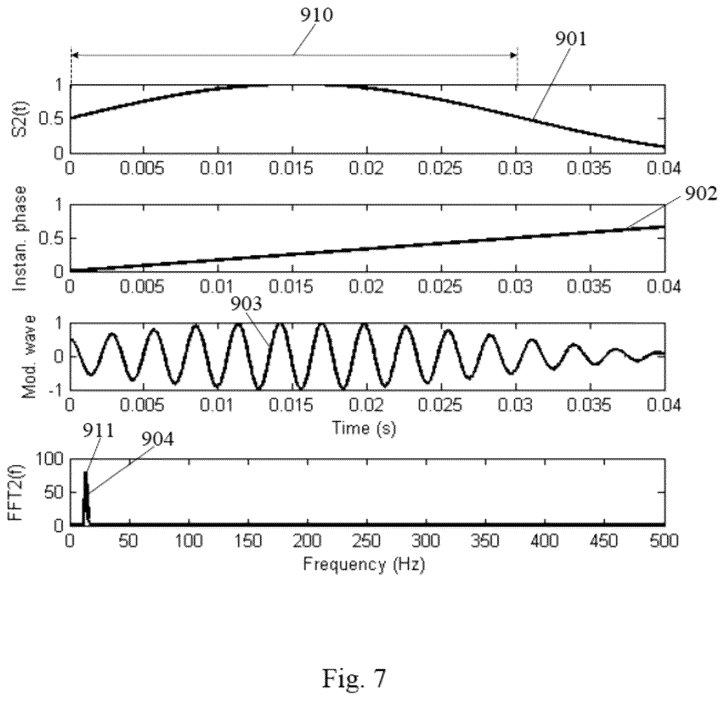

FIG. 7 illustrates another example of various waveforms according to the present disclosure. In this example, both the first and second modulating signals are assumed to be sinusoidal. The horizontal axes of waveforms 901, 902, and 903 represent time (seconds), while the horizontal axis of waveform 904 represents frequency (Hz). In the waveform 901, a modulating signal S2(t) with a first temporal modulation frequency of 16.4 Hz is shown. A half-period interval 910 corresponds to this frequency. The instantaneous phase of S2(t), depicted in the waveform 902, is used to modulate a second signal at 19.1 Hz, resulting in a phase-amplitude modulated waveform shown in the waveform 903. Upon demodulation, a cross-frequency modulation component at 13.7 Hz may emerge, as shown by a frequency spike 911 in the FFT2(f) spectrum, shown in the waveform 904. In this case, the brain may perceive a composite modulation pattern and demodulates it into an effective alpha-band signal (13.7 Hz). This perceptually benign alpha rhythm can entrain neural activity. Moreover, the entrained alpha rhythm may induce or modulate gamma oscillations via endogenous alpha-gamma cross-frequency coupling, which is known to stimulate more complex or resonant neural circuits. This effect reflects a deeper level of neural engagement, beyond mere flicker perception. Notably, human SSVEP responses are typically strongest within the 6-15 Hz fundamental frequency range, indicating that alpha-band stimulation is particularly effective for visual entrainment.

Whereas preferred embodiments of the present disclosure have been described and illustrated, it will be understood that various alterations, modifications, and improvements may be made without departing from the scope of the appended claims. For example, alternative LED driving circuits—with output voltage and current modulation—may be embedded within LED luminaires using different combinations of components to achieve the same or similar objectives as disclosed herein. Accordingly, the foregoing description and accompanying drawings are intended for illustrative purposes only and should not be construed as limiting the scope of the disclosure.

What is claimed is:

1. A light-emitting diode (LED) luminaire with dual-frequency flickers, comprising:
a first set of LED arrays and a second set of LED arrays, each having a forward voltage;
an LED printed circuit board with a light diffuser disposed in front of the LED printed circuit board;
at least one power supply unit configured to receive an alternating current (AC) line voltage from a mains source and convert it into a primary direct-current (DC) voltage greater than the forward voltage;
at least one multi-channel pulse-width modulation (PWM) pulse generator configured to generate a first modulating signal at a first predetermined temporal modulation frequency, and to generate a second modulating signal at a second predetermined temporal modulation frequency higher than the first predetermined temporal modulation frequency;
an LED driving circuit comprising at least two modulation circuits, each configured to generate a first driving current and a second driving current in response to the first modulating signal and the second modulating signal, respectively, to drive the first set of LED arrays and the second set of LED arrays; and
a positive voltage regulator configured to convert the primary DC voltage into a secondary DC voltage that is positive relative to a ground reference to operate the at least one multi-channel PWM pulse generator to generate the first and second modulating signals to modulate the first and second driving currents,
wherein:
each of the first set of LED arrays and the second set of LED arrays comprises a plurality of LED arrays arranged respectively in two interlaced fields, one occupying odd-numbered rows and the other occupying even-numbered rows;
the plurality of LED arrays of the first set of LED arrays and the second set of LED arrays are mounted on the LED printed circuit board and configured to collectively emit a combined light, which is optically blended by the light diffuser to produce a uniform illumination, thereby visually unifying the dual-frequency flickers emitted from the plurality of LED arrays in the odd-numbered and even-numbered rows into a single perceived field; and
each of the at least two modulation circuits is configured to control the plurality of LED arrays in the odd-numbered and even-numbered rows to turn on or off in response to the first modulating signal and the second modulating signal.

2. The light-emitting diode (LED) luminaire with dual-frequency flickers of claim 1,
wherein the primary DC voltage is applied to both the first set of LED arrays and the second set of LED arrays relative to the ground reference.

3. The light-emitting diode (LED) luminaire with dual-frequency flickers of claim 1,
wherein each of the at least two modulation circuits comprises a first terminal and a second terminal, the first terminal being configured to be coupled to negative terminals of both the first set of LED arrays and the second set of LED arrays, and the second terminal being configured to be coupled to the ground reference.

4. The light-emitting diode (LED) luminaire with dual-frequency flickers of claim 1,
wherein each of the first and second modulating signals comprises PWM pulses having a duty cycle of at least 40% and a predetermined period, wherein each of the first and second driving currents incorporates the respective first and second modulating signals, each associated with its corresponding predetermined period, and wherein the combined light emitted from the first and second sets of LED arrays exhibits the respective predetermined periods in response to the first and second driving currents.

5. The light-emitting diode (LED) luminaire with dual-frequency flickers of claim 4,
wherein, upon absence of either the first LED driving current or the second LED driving current, the other driving current is configured to increase a duty cycle to at least 75%, thereby maintaining a steady combined light output and minimizing perceptible flicker.

6. The light-emitting diode (LED) luminaire with dual-frequency flickers of claim 1,
wherein both the first and second predetermined temporal modulation frequencies are higher than a critical flicker frequency (CFF), such that no flicker is perceptible to human eyes, and wherein the flickers remain detectable by a human brain for visual stimulation.

7. The light-emitting diode (LED) luminaire with dual-frequency flickers of claim 6,
wherein the first predetermined temporal modulation frequency is nominally 80 Hz and the second predetermined temporal modulation frequency is nominally 120 Hz, wherein the combined light emitted from the first and second sets of LED arrays produces visual stimulation flickers at both 80 Hz and 120 Hz, and wherein the flickers are configured to induce a symmetric harmonic at 40 Hz in a human brain, thereby endogenously entraining gamma-band neural oscillations.

8. The light-emitting diode (LED) luminaire with dual-frequency flickers of claim 7,
wherein the visual stimulation flickers at 80 Hz are configured to induce a subharmonic at 40 Hz in a human brain, thereby endogenously entraining the gamma-band neural oscillations, and wherein dual entrainment is achieved through both beat frequencies and harmonic coupling, thereby enabling flexible and targeted neuromodulation.

9. The light-emitting diode (LED) luminaire with dual-frequency flickers of claim 1,
wherein each of the at least two modulation circuits comprises an electronic switch configured to be selectively turned on or off, thereby controlling the plurality of LED arrays in response to the first modulating signal and the second modulating signal.

10. The light-emitting diode (LED) luminaire with dual-frequency flickers of claim 9,
wherein each of the at least two modulation circuits further comprises a respective resistor network configured to provide biasing for the corresponding electronic switch, thereby facilitating the generation of the first and second LED driving currents with a high signal-to-noise ratio.

11. The light-emitting diode (LED) luminaire with dual-frequency flickers of claim 9,
wherein each electronic switch comprises either a bipolar junction transistor (BJT) or a metal-oxide-semiconductor field-effect transistor (MOSFET).

12. The light-emitting diode (LED) luminaire with dual-frequency flickers of claim 1,
wherein each of the first set of LED arrays and the second set of LED arrays further comprises a plurality of current-limiting resistors configured to respectively couple to the plurality of LED arrays and to prevent thermal runaway or destruction of the plurality of LED arrays from excess current.

13. The light-emitting diode (LED) luminaire with dual-frequency flickers of claim 1,
wherein an application of the second driving current to the second set of LED arrays is temporally offset from an application of the first driving current to the first set of LED arrays, thereby introducing a time delay between the first and second modulating signals.

14. The light-emitting diode (LED) luminaire with dual-frequency flickers of claim 1,
wherein the at least one multi-channel PWM pulse generator comprises a human interface configured to receive user-defined commands to select combinations of modulation frequencies and tuning parameters, thereby enabling personalized modulation of brain rhythms, enhancing neural synchronization and entrainment, and targeting specific cognitive states or neurological biomarkers for therapeutic purposes.

15. The light-emitting diode (LED) luminaire with dual-frequency flickers of claim 1,
wherein the first predetermined temporal modulation frequency is nominally 16.4 Hz, and the second predetermined temporal modulation frequency is nominally 19.1 Hz, wherein the combined light emitted from the first and second sets of LED arrays produces dual-frequency flickers at both 16.4 Hz and 19.1 Hz for visual stimulation, and wherein the dual-frequency flickers are configured to induce a symmetric harmonic at nominally 13.7 Hz in a human brain, thereby endogenously enhancing alpha rhythms and facilitating subsequent entrainment of gamma oscillations.

16. The light-emitting diode (LED) luminaire with dual-frequency flickers of claim 1,
wherein the at least one multi-channel PWM pulse generator comprises a microcontroller.
